(19) United States
(12) Patent Application Publication
(10) Pub. No.: US 2002/0160407 A1
(43) Pub. Date: Oct. 31, 2002

(54) Method of diagnosing and treating pneumonia and pneumonia-related sepsis associated with the IL6-174 gene polymorphism

(76) Inventors: Grant William Waterer, Yokine (AU); Richard Glenn Wunderink, Germantown, TN (US)

Correspondence Address: Licata & Tyrrell P.C., 66 E. Main Street, Marlton, NJ, 08053, US

(21) Appl. No.: 10/114,397

(22) Filed: Apr. 1, 2002

Related U.S. Application Data
(60) Provisional application No. 60/281,227, filed on Apr. 3, 2001.

(51) Int. Cl. C12Q 1/68; C07H 21/04

(52) U.S. Cl. 435/6; 536/23.5

(57) ABSTRACT

Methods and compositions for diagnosing diseases associated with a genetic polymorphism in an IL6 gene via determination of the genotype of an IL6 gene at the -174 site are provided. The methods of this invention can be used to diagnose diseases such as pneumonia and septic shock or to diagnosis a predisposition or susceptibility to pneumonia and pneumonia-related sepsis. Also related are methods for the treatment of pneumonia or predisposition or susceptibility to pneumonia or pneumonia-related sepsis. Compositions are also provided for identifying the genetic polymorphism.

This application claims the benefit of priority from U.S. provisional application Serial No. 60/281,227, filed Apr. 3, 2001.

### INTRODUCTION

1. Field of the Invention

This invention relates to diagnostic methods based upon the identification of a polymorphism in the Interleukin 6 (“IL6”) gene, more specifically, a Guanine (“G”) to Cytosine (“C”) transition at position -174 within the promoter region of the IL6 gene wherein the G allele is associated with greater IL6 gene transcription and IL6 production. This invention also relates to a method for diagnosis of pneumonia, and a method of identifying and diagnosing pre-disposition for septic shock in patients with pneumonia. The invention further relates to compositions for screening for the polymorphism and improved treatment choices for patients diagnosed with pneumonia or diagnosed as being susceptible to pneumonia or pneumonia-related sepsis by the methods of the present invention.

2. Background of the Invention

Pneumonia is a common clinical entity, particularly among the elderly. Pneumonia is a major health problem worldwide. Pneumonias are classified as being either community-acquired or hospital-acquired (nosocomial). In the United States, Community Acquired Pneumonia (CAP) is the leading cause of death due to infection and the sixth most common cause of death overall. Further, nosocomial pneumonia or hospital-acquired pneumonia, defined as pneumonia occurring more than 48 hours after admission to the hospital is a major cause of morbidity and mortality in hospitalized patients. Nosocomial pneumonia is the second most common cause of hospital acquired infection and has a mortality rate of about thirty percent.

A thorough understanding of the epidemiology and microbiology of pneumonia is essential for appropriate diagnosis and management. Although the microbiology of pneumonia has remained relatively stable over the last decade, there is new information on the incidence of a typical pathogens, and new information on the incidence of pathogens in cases of CAP and in CAP in the elderly. Recent studies have provided new data on risk factors for mortality in CAP, which can assist the clinician in making decisions about the need for hospital admission. The emergence of antimicrobial resistance in Streptococcus pneumonia, the organism responsible for most cases of CAP, has greatly affected the approach to therapy, especially in those patients who are treated empirically. Guidelines for the therapy of CAP have been published by the American Thoracic Society, the British Thoracic Society, and, most recently, the Infectious Diseases Society of America and others.

Treatment of nosocomial pneumonia, like treatment of CAP is usually empiric. Because of the high mortality rate of nosocomial pneumonia, therapy must be started as soon as pneumonia is suspected. The most common organisms responsible for nosocomial pneumonia are Pseudomonas aeruginosa, Staphylococcus aureus, Enterobacter, Klebsiella pneumoniae, and Escherichia coli.

Pneumonia remains a significant health problem and patients continue to die despite receiving appropriate antibiotic therapy. Modification of the host immune response has yet to live up to the promise of improved outcome. Despite this, there is significant reason for optimism. Some immunomodulatory therapies clearly have efficacy in some patients. As the understanding of the immune response to pneumonia improves, the ability to tailor specific therapies for individual patients will also improve, hopefully avoiding the deleterious effects that have so far prevented the development of an effective immune-based therapy. The possibility of delivering cytokines directly to the lung, is a particularly promising way of achieving the desired pulmonary effect without systemic side effects. Corticosteroids are currently unique in that they have a proven role in the therapy of pneumonia due to P. carinii.

Once respiratory failure has ensued, supportive measures such as patient positioning and differential lung ventilation can improve oxygenation at no additional risk in some patients, particularly those with severe unilateral pneumonia. In facilities where Extracorporeal Membrane Oxygenation (ECMO) is available it may be beneficial in selected patients when all other means of providing respiratory support have failed.

The past twenty years have seen an explosion in the knowledge of human immunology, with exploration of the therapeutic possibilities just beginning. The next decade may finally provide a significant advance in the therapy of pneumonia, the first substantial gain since penicillin.

In light of the prevalence of pneumonia and the evolution of resistance in the most common bacterial pneumonia pathogens, physicians advise obtaining specimens for culture of pathogens and analysis of patterns of susceptibility, especially of S. pneumonia, in their communities; using antibiotics appropriately and prudently, according to prevailing susceptibilities when empirical treatment is called for; and immunizing susceptible patients with pneumococcal and influenza vaccines. This is because the mortality of patients with severe pneumonia continues to increase (Fine et al. 1997 New Engl. J. Med.;336:243-250, 1987 British Thoracic Society, Q. J. Med. 239:192-220, Niederman et al. 1993 Am. Rev. Resp.Dis. 148:1418-1426). In such cases an ability to improve accuracy of diagnosis of pneumonia, would be of distinct advantage and may lead to improved outcomes and lower medical costs for such patients.

IL6 is a cytokine which is released in large amounts during the immune response to pneumonia. It has predominantly pro-inflammatory action, in that it increases a patient's inflammatory response. Glynn et al. 1999, Thorax 54:51-55. Pneumonia is the most common cause of severe sepsis. High circulating levels of IL-6 have been detected in patients with septic shock. Calandra et al. 1991, Am J. Medicine 91:23-29. IL6 has also been found to play a role in other diseases, such as juvenile chronic arthritis, myasthenia gravis, and lupus. Fishman et al. 1998, J. Clin Invest. 102:1369-1376.

It is a surprising feature of the present invention to be able to diagnose the presence of pneumonia and pneumonia-related sepsis, and the predisposition of a patient to acquire pneumonia or pneumonia-related sepsis by the methods of the present invention.

### BRIEF SUMMARY OF THE INVENTION

It is an object of the present invention to improve diagnosis of pneumonia and pneumonia-related sepsis by identification of a polymorphism of the IL6 gene at the -174 site. It is a particular object of the invention to provide a method of diagnosis of a patient's predisposition or susceptibility to pneumonia and pneumonia-related sepsis. A further object is to provide, following such diagnosis, a method of identifying patients for alternative management of pneumonia before the disease becomes significantly established or results in pneumonia-related sepsis. Thus, the invention also relates to compositions for screening for the IL6-174 polymorphism site and improved treatment choices for patients diagnosed with pneumonia and/or pneumonia-related sepsis or diagnosed as being susceptible to pneumonia or pneumonia-related sepsis by the methods of the present invention.

Other preferred embodiments of the present invention will be apparent to one of ordinary skill in light of the following description of the invention and of the claims.

### DETAILED DESCRIPTION OF THE INVENTION

In a first aspect, the invention provides a method of diagnosing a disease associated with a genetic polymorphism (Cytosine (“C”) at position -174 (Olomolaiye et al. 1997 Immunology 92:66; Olomolaiye et al. 1998 Eur.J. Immunogenet. 25(2):267, Fishman et al. 1998 J.Clin. Invest.102(7):1369-76) in the promoter region of the IL6 gene rather than the usual Guanine (“G”)) in an animal predisposed or susceptible to said disease, by determining the genotype of IL6 in said animal. The term animal is meant to include humans. The first aspect of the invention further provides a method of identifying an animal predisposed or susceptible to a disease associated with a genetic polymorphism in the IL6 gene, by determining the genotype of said IL6 gene in said animal. In an embodiment of the invention, the method of diagnosis is to screen for an individual at risk of a condition or disease such as pneumonia or pneumonia-related sepsis correlated with an IL6 gene polymorphism (“C”) at the -174 site.

The invention is based upon the identification of a correlation between polymorphisms in the IL6 gene, specifically at the -174 position, and a predisposition or susceptibility to pneumonia and/or pneumonia-related sepsis. It has been found that an IL6 gene polymorphism of a G to C transition at the -174 site is linked with pneumonia and pneumonia-related sepsis. The polymorphism is located within the promoter region of the IL6 gene and the G allele is associated with greater IL6 gene transcription and IL6 production. Pneumonia is the most common cause of severe pneumonia-related sepsis. Serum IL6 levels have been found to correlate with the outcome from pneumonia-related sepsis, namely the higher the serum level, the greater the mortality of the patients. It appears that patients genetically programmed to respond with greater IL6 response, i.e. those possessing the G to C transition of the IL6-174 site, may be at greater risk of severe pneumonia-related sepsis. Severe pneumonia-related sepsis includes septic shock, respiratory failure and death. The present invention is of advantage in that by screening for the presence of the polymorphism it is possible to identify individuals likely to have a genetic predisposition or susceptibility to pneumonia and pneumonia-related sepsis. It may also result in substantially different management, both prevention and treatment, if pneumonia occurs, with subsequent substantial improvement in mortality and morbidity from pneumonia or pneumonia-related sepsis.

In an embodiment of the invention, diagnosis is carried out by determining whether an IL6 gene contains a polymorphism “C” at position -174. Possessing a fragment that contains the “C” at that -174 site correlates with increased risk of predisposition or susceptibility to pneumonia and pneumonia-related sepsis. As a human genome contains two IL6 genes, one on each of a pair of chromosomes, an individual can accordingly be found to be homozygous or heterozygous for the risk polymorphism, or to lack the risk polymorphism.

Genotypic and allelic frequencies of this invention are readily determined by a number of methods known to those skilled in the art. Methods used in the present invention include using PCR amplification and restriction enzyme digestion. The method conveniently comprises amplifying a fragment of an IL6 gene to produce copies and determining whether copies of the fragment contain the polymorphism.

Another suitable technique is to amplify the fragment using PCR techniques, producing copies of a fragment that is at least 500 base pairs in length, preferably at least 600 base pairs in length. It is preferred that the PCR primers are selected so as to amplify a region of the gene that is about 740 base pairs in length. PCR techniques are well known in the art and it would be within the ambit of a person of ordinary skill in this art to identify primers for amplifying a suitable section of the IL6 gene. PCR techniques are described for example in EP-A-0200362 and EP-A-0201 184. In a further embodiment of the invention, the diagnostic method comprises analysis of the IL6 gene using single strand conformational polymorphism (SSCP) mapping to determine whether the IL6 gene is the risk or the non-risk form.

In preferred embodiments, the method comprises screening an IL6 gene, and this screening is conveniently carried out by any one of a number of suitable techniques that are known in the art, and may be conveniently selected from amplification of a nucleic acid sequence located within the IL6 gene, Southern blotting of regions of the gene and single strand conformational polymorphism mapping of regions within the gene. The genotype in that region is also optionally determined using hybridization probes adapted selectively to hybridize with the particular polymorphism of the IL6 gene at the -174 location which is associated with predisposition or susceptibility to disease. A probe used for hybridization detection methods must be in some way labeled so as to enable detection of successful hybridization events. This is optionally achieved by in vitro methods such as nick-translation, replacing nucleotides in the probe by radioactively labeled nucleotides, or by random primer extension, in which non-labeled molecules act as a template for the synthesis of labeled copies. Other standard methods of labeling probes so as to detect hybridization are known to those skilled in this art.

According to a second aspect of the invention, there is provided a method of diagnosis and therapy comprising diagnosing a predisposition or susceptibility to pneumonia and pneumonia-related sepsis according to the methods of the invention and treating or managing the treatment of an individual having a predisposition or susceptibility to pneumonia and pneumonia-related sepsis to prevent or slow the onset of pneumonia and pneumonia-related sepsis.

Known therapies for pneumonia can be effective in halting advancement of the disease, or at least slowing the advancement. IL6 gene analysis may also lead to more appropriate placement of patients into intensive care/critical care units, an important factor in optimizing survival from pneumonia. It is thus an advantage of the invention that early diagnosis of susceptibility to pneumonia and pneumonia-related sepsis is improved, so that preventative therapy can be started as soon as possible, optimizing any interventions potential (such as immunomodulatory therapy) for affecting outcome. As alternative diagnostic methods improve and are developed, the present invention can add to the total knowledge of the risk of developing pneumonia and pneumonia-related sepsis in an individual. The decision of a physician on how and whether to initiate therapy in anticipation of the disease can be taken with increased confidence.

A variety of suitable treatments of pneumonia and pneumonia-related sepsis are described in the art. See e.g., Hirani and MacFarlane Thorax 1997;52:17-21, Pachon J et al. Am. Rev. Resp. Dis. 1990;142:369-373, Ruiz M et al. Am. J. Respir. Crit. Care. Med. 1999;160:923-929, Leeper and Torres Clin. Chest. Med. 1995;16:155-171. Other treatments are known to persons of skill in the art.

Another aspect of the invention provides a composition for use in diagnosing a disease associated with a genetic polymorphism in an IL6 gene in an animal predisposed or susceptible to said disease, said composition comprising one or more primer nucleic acid molecules adapted to amplify a portion of an IL6 gene selected from a portion of the gene around the -174 location.

Another aspect of the invention also provides a composition for use in identifying an animal predisposed or susceptible to a disease associated with a genetic polymorphism in an IL6 gene, said compositions comprising one or more primer nucleic acid molecules adapted to amplify a portion of the IL6 gene selected from a portion of the gene around the -174 location.

The compositions of this aspect of the invention may comprise a nucleic acid molecule capable of identifying the -174 polymorphism in said IL6 gene, said polymorphism being indicative of a risk genotype in said animal.

A further embodiment of the invention provides a composition for diagnosis of pneumonia or pneumonia-related sepsis, or predisposition or susceptibility to pneumonia or pneumonia-related sepsis, comprising means for determining the genotype of an IL6 gene in an individual, for example whether an individual is homozygous or heterozygous for polymorphic variants of an IL6 gene at the -174 location.

In an embodiment of the invention, a diagnostic composition comprises PCR primers adapted to amplify a DNA sequence within and around the IL6-174 polymorph location, wherein alternative versions of the gene are distinguished one from another.

In a further aspect of the invention there is provided a diagnostic kit comprising a diagnostic composition as described above and an indicator composition for indicating how possessing a polymorphic version of an IL6 gene correlates with the presence or predisposition to pneumonia and/or pneumonia-related sepsis.

Diagnostic kits are typically accompanied by or comprise a chart or other visual aid for assistance in interpreting the results obtained using the kit. Suitable indicator compositions for use in the diagnostic kit of the invention include a leaflet or other visual reminder that possessing the risk polymorphism version of an IL6 gene correlates with pneumonia or pneumonia-related sepsis or an increased risk of a predisposition or susceptibility to pneumonia and/or pneumonia-related sepsis.

In a still further aspect of the invention there is provided a means for diagnosing whether an individual has a predisposition or susceptibility to pneumonia, using PCR primers adapted to amplify a region around -174 region in the IL6 gene. Alternative versions of the gene are typically distinguished one from another by means known to those skilled in the art.

Multiple techniques exist and are known to one skilled in the art in the manufacture of means for diagnosing whether an individual has a predisposition or susceptibility to pneumonia and/or pneumonia-related sepsis, by PCR primers adapted to amplify a region around the -174 position in the IL6 gene. Restriction analysis can be employed, where the enzyme cuts if a “G” is present, but not if an “C” is present, and when run on a gel, e.g., 1% agarose gel, the different fragments migrate differently based upon size.

According to the invention, an individual who is homozygous for a risk polymorphism, that is to say homozygous for a version of -174 (“CC”), is classified as being at highest risk of pneumonia or predisposition or susceptibility to pneumonia and/or pneumonia-related sepsis. An individual being heterozygous (“GC”) is classified as having moderate risk of pneumonia and/or pneumonia-related sepsis; or a predisposition or susceptibility to pneumonia or pneumonia-related sepsis.

Optionally, the assessment of an individual's risk factor according to any aspect of the invention is calculated by determining the genotype of an IL6 gene polymorphism and combining the result with analysis of other known genetic, physiological, dietary, clinical, or other indications known to those of skill in the art. The invention in this way provides further information upon which measurement of an individual's risk can be based.

While, it is possible that IL6 polymorphisms are not the disease causing genes, nevertheless, the observed correlation is of use in diagnosis of pneumonia and pneumonia-related sepsis and assessment of the risk of predisposition or susceptibility to pneumonia and pneumonia-related sepsis.

In another embodiment of the invention, the results of the genotyping done herein are used, along with other diagnostic measures and disease parameters, by treatment providers to determine the best course of treatment for the patient having pneumonia or having been determined as susceptible to pneumonia and/or pneumonia-related sepsis by the methods of this invention.

In another aspect of the invention, a method of identification and diagnosis of patients with pneumonia at an increased risk of septic shock is provided. A significant association between the IL6-174 genotype and the risk of septic shock in patients with pneumonia has been identified.

In an effort to identify patients at high risk for subsequent septic shock, the systemic inflammatory response syndrome (SIRS) criteria were developed. SIRS is defined as having at least two of the following four conditions: (1) oral temperature of >38 C. or <36 C.; (2) respiratory rate of >20 breaths/minute or Pa_{co2} or <32 count torr; (3) heart rate of >90 beats/minute; (4) leukocyte count of >12,000/μl or <4,000/μl or >10% bands. Hypoxemia was included as an organ dysfunction and is one of the two most common organ dysfunctions in most studies of SIRS. Hypoxemia (and other organ dysfunctions in SIRS) is assumed to occur by the same mechanism and reflect the same pro-inflammatory state as septic shock.

Clearly non-genetic factors such as the length of time to initial therapy and adequacy of therapy also play important roles in the ultimate development of the clinical presentation, including the development of pneumonia-related sepsis. However, this present invention supports the hypothesis that genetic polymorphisms play a critical role in the variable presentation of pneumonia and pneumonia-related sepsis. Differences in the virulence of pathogens also has an impact on outcome of pneumonia. These experiments did not analyze the influence of genotype on pneumonia for individual pathogens due to the low number of cases with a definite microbiological diagnosis. The problem of the low sensitivity of traditional culture techniques in pneumonia is well known, and the diagnostic rate observed is not inconsistent with studies of the yield of cultures in patients with pneumonia. However, the pattern in patients with proven or suspected pneumonia parallels that of the entire cohort, a finding which is not surprising given that the pneumococcus is the most common cause of pneumonia. Septic shock is associated with the G to C polymorphism at the -174 locus of the IL6 gene.

It will be readily apparent to one of ordinary skill in the relevant arts that other suitable modifications and adaptations to the methods and applications described herein are obvious and may be made without departing from the scope of the invention or any embodiment thereof.

What is claimed is:

1. A method of diagnosing pneumonia associated with a genetic polymorphism in an IL6 gene at the -174 locus in an animal, said method comprising determining the genotype of said IL6 gene in said animal, and diagnosing pneumonia based on said genotype.

2. A method of identifying an animal predisposed or susceptible to pneumonia associated with a genetic polymorphism in an IL6 gene at the -174 locus, said method comprising determining the genotype of said IL6 gene in said animal, and identifying said animal based on said genotype.

3. A method of diagnosing a predisposition to pneumonia-related sepsis associated with a genetic polymorphism in an IL6 gene at the -174 locus in an animal, said method comprising determining the genotype of said IL6 gene in said animal, and diagnosing a predisposition to pneumonia-related sepsis based on said genotype.

4. A method of diagnosis of a predisposition or susceptibility to pneumonia or pneumonia-related sepsis in an individual, the method comprising determining whether the individual possesses a polymorphic risk version of an IL6 gene at the -174 locus, a polymorphic risk version of the gene being one that has an C at site -174, the method comprising:
(a) using restriction analysis with an enzyme capable of cutting the IL6 gene to determine if a Guanine is present at the -174 site and not a Cytosine.
(b) testing whether the copies contain Guanine or a Cytosine at site -174 through known differences in how such fragments migrate on a gel, and thereby determining whether the individual is homozygous or heterozygous for a polymorphic risk version of the gene, and
(c) diagnosing the individual's predisposition or susceptibility to pneumonia or pneumonia-related sepsis as greatest if that individual is homozygous for the polymorphic risk version of the gene at the -174 site, moderate if that individual is heterozygous for the polymorphic risk version at the -174 site, and least if that individual lacks the polymorphic risk version at the -174 site.

5. A method of managing and treating patients with a predisposition to pneumonia or pneumonia-related sepsis comprising, determining whether the individual possesses a polymorphic risk version of an IL6 gene at the -174 locus, a polymorphic risk version of the gene being one that has a Cytosine at site -174, wherein a patient having such polymorphism is managed and treated as if said patient has pneumonia or pneumonia-related sepsis or is predisposed or susceptible to pneumonia or pneumonia-related sepsis.

6. A method of diagnosing patients with a predisposition or susceptibility to pneumonia or pneumonia-related sepsis comprising determining whether the patient possesses a polymorphic risk version of an IL6 gene wherein the polymorphic risk version has a Cytosine in the -174 locus.

7. A method of diagnosing patients with pneumonia at an increased risk of septic shock comprising determining whether the patient possesses a polymorphic risk version of the IL6 gene wherein the polymorphic risk version has a Cytosine in the -174 site.

8. A composition for use in identifying an animal predisposed or susceptible to a disease associated with a genetic polymorphism in an IL6 gene, comprising a primer nucleic acid molecule adapted to amplify a portion of the IL6 gene selected from a portion of the gene around the -174 site location.
